(19) United States
(12) Patent Application Publication
(10) Pub. No.: US 2012/0040914 A1
(43) Pub. Date: Feb. 16, 2012

(54) ENHANCING EFFECTIVENESS OF GLIAL CANCER THERAPIES

(75) Inventors: MEHARVAN SINGH, Benbrook, TX (US); Adrienne Badeaux, Fort Worth, TX (US); Niki Mirshams, Frisco, TX (US)

(73) Assignee: UNIVERSITY OF NORTH TEXAS HEALTH SCIENCE CENTER AT FORT WORTH, Fort Worth, TX (US)

(21) Appl. No.: 13/207,995

(22) Filed: Aug. 11, 2011

Related U.S. Application Data
(60) Provisional application No. 61/372,620, filed on Aug. 11, 2010.

(51) Int. Cl. A61K 38/00; A61P 35/00; A61K 31/70; A61K 31/56; A61K 31/7088

(52) U.S. Cl. 514/19.3; 514/171; 514/19.2; 514/44 R; 514/23

(57) ABSTRACT

A method of enhancing the effectiveness of a chemotherapeutic agent, such as Temozolomide (TMZ), or ionizing radiation against glioblastoma is described in the present invention. The method comprises targeting putative membrane androgen receptor (termed mAR) in glioma cells to enhance the cytotoxic effects of a chemotherapeutic agent or ionizing radiation while simultaneously affording protection to neighboring neurons.

### CROSS-REFERENCE TO RELATED APPLICATION

This application claims the benefit of U.S. Provisional Application Ser. No. 61/372,620, filed Aug. 11, 2010, the disclosure of which is hereby incorporated by reference in its entirety, including all figures, tables and amino acid or nucleic acid sequences.

### STATEMENT OF FEDERALLY FUNDED RESEARCH

This invention was made with U.S. Government support under Contract No. AG026672 awarded by the National Institutes of Health (NIH). The government has certain rights in this invention.

### TECHNICAL FIELD OF THE INVENTION

The present invention relates in general to the field of anti-cancer therapy, and more particularly, to a method of enhancing the effectiveness of chemotherapy against glial cancer while protecting neurons by targeting the membrane androgen receptor.

### BACKGROUND OF THE INVENTION

Without limiting the scope of the invention, its background is described in connection with membrane androgen receptors (mAR) and tumor therapy.

United States Patent Application No. 20100048676 (Chang, 2010) discloses compositions and methods for modulating androgen receptor (AR) activity, such as non-androgen dependent AR activity. Also disclosed are compositions and methods for diagnosing beast cancer and for inhibiting liver cancer growth. In addition, disclosed are methods for identifying molecules that inhibit AR in non-androgen dependent ways.

United States Patent Application No. 20070141581 (Singh and Gatson, 2007) includes compositions, kits and methods for specifically and differentially activating a membrane androgen receptor and their use for comparing the binding specificity of one or more drugs to a membrane androgen receptor and to an intracellular androgen receptor, wherein a difference in drug binding is indicative of differential receptor binding and may be used to diagnose and treat diseases and conditions associated with androgens.

United States Patent Application No. 20080267875 (Castanas, 2008) describes conjugates comprising one or more steroids conjugated with one or more mammalian proteins. The conjugates are useful for diagnosis or treatment of solid cancer and hematological malignancies. Further the conjugates exhibit a synergistic action together with a cytoskeleton acting drug such as Taxol®, which enables the treatment of cancers that otherwise would be non responsive to Taxol®.

### SUMMARY OF THE INVENTION

The present invention discloses methods and compositions for targeting the membrane androgen receptor to enhance the effectiveness of the chemotherapeutic agents and/or radiation therapy against glioma. The strategy of targeting the membrane androgen receptor (mAR) as described herein not only increases the vulnerability of glial tumor cells to existing chemotherapeutic agents and/or ionizing radiation but also promotes the survival/viability of surrounding healthy neurons (neuronal cells) from the toxic consequences of the chemotherapeutic agents.

The present invention in one embodiment provides a composition for enhancing simultaneously the effectiveness of one or more chemotherapeutic agents and/or radiation therapy and for protecting one or more brain cells, neurons or both, wherein the chemotherapeutic agents treat, ameliorate symptoms, or delay a progression of one or more gliomas comprising: one or more chemotherapeutic agents selected from the group consisting of dacarbazine alkylating agents, salinomycin, temozolomide, procarbazine, nitrosoureas, bis-chloronitrosourea, lomustine, and platinum based chemotherapeutic agents, one or more membrane androgen receptor (mAR) activating agents, agonists or both, wherein the agents are selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene, and one or more optional pharmaceutically acceptable excipients. In one aspect the one or more gliomas are selected from the group consisting of astrocytomas, ependymal tumors, glioblastoma multiforme, and primitive neuroectodermal tumors. in another aspect the mAR activating agent is selected from a testosterone or a dihydrotestosterone and can also include synthetic derivatives of testosterone comprising testosterone propionate, testosterone cypionate, and fluoxymesterone.

In yet another aspect the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle. In a specific aspect the conjugating agent is a protein selected from a bovine serum albumin or a human serum albumin. In another aspect the composition is administered orally, intravenously, intramuscularly, subcutaneously, intracranially or by any other suitable parenteral route. In another aspect the one or more chemotherapeutic agents are administered in a dose ranging from 10 μM-10 mM or from 5 μM to 10 mM (salinomycin) and the one or more mAR activating agents or agonists are administered in a dose ranging from 1 nM-10 μM.

In another embodiment the instant invention provides a method of treating, ameliorating symptoms, delaying progression or combinations thereof of one or more glial cancers in a subject comprising the steps of: identifying the subject in need of the treatment, amelioration of the symptoms, delaying the progression or combinations thereof of the glial cancers and administering a therapeutically effective amount of a pharmaceutical composition sufficient to treat, ameliorate symptoms, delay progression or combinations thereof of the one or more cancers in the subject comprising: (i) one or more chemotherapeutic agents, wherein the one or more chemotherapeutic agents are selected from the group consisting of dacarbazine alkylating agents, salinomycin, temozolomide, procarbazine, nitrosoureas, bis-chloronitrosourea, lomustine, and platinum based chemotherapeutic agents, (ii) one or more membrane androgen receptor (mAR) activating agents, agonists or both wherein the agents are selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene, and (iii) one or more optional pharmaceutically acceptable excipients, wherein the composition simultaneously kills one or more glial cancer cells and protects one or more brain cells, neurons or both. Optionally, the method also provides for the application of radiation therapy to the subject for treating, ameliorating symptoms, delaying progression or combinations thereof of one or more glial cancers.

The glial cancers that can be treated by the method presented hereinabove are selected from the group consisting of astrocytomas, ependymal tumors, glioblastoma multiforme, and primitive neuroectodermal tumors. In one aspect of the method the mAR activating agent, agonist or both are selected from a testosterone or a dihydrotestosterone. In another aspect the synthetic derivatives of testosterone comprise testosterone propionate, testosterone cypionate, and fluoxymesterone. In yet another aspect the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle. Specifically, the conjugating agent is a protein selected from a bovine serum albumin or a human serum albumin.

In one aspect of the method the composition is administered orally, intravenously, intramuscularly, subcutaneously, intracranially or by any other suitable parenteral route. In another aspect the one or more chemotherapeutic agents are administered in a dose ranging from 10 μM-10 mM or from 5 μM-10 mM (salinomycin). In yet another aspect the one or more mAR activating agents or agonists are administered in a dose ranging from 1 nM-10 μM.

In yet another embodiment the instant invention discloses a therapeutic composition comprising one or more membrane androgen receptor (mAR) activating agents, agonists or both in an amount sufficient to enhance a cytotoxic activity of one or more chemotherapeutic agents against one or more glioma cells by a suppression of one or more cell signaling effectors, pathways or both, wherein the composition simultaneously kills the one or more glioma cells and protects one or more brain cells, neurons or both. In a specific aspect the chemotherapeutic agent is temozolomide, salinomycin or a combination of temozolomide and salinomycin. In one aspect the one or more mAR activating agents or agonists selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene. In another aspect the mAR activating agent is selected from a testosterone or a dihydrotestosterone. In yet another aspect the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle. In one aspect the conjugating agent is a protein selected from a bovine serum albumin or a human serum albumin. In another aspect the composition suppresses methylguanine methyltransferase (MGMT) activity, PI3K/Akt activity, extracellular signal regulated kinases (ERKs) or combinations thereof in the one or more glioma cells.

The present invention also describes a method for enhancing simultaneously the efficacy of a chemotherapy and/or radiation therapy and for protecting one or more brain cells, neurons or both in a subject comprising the steps of: identifying the subject suspected of having a need for the treatment of a glioma and administering one or more membrane androgen receptor (mAR) activating agents, agonists or both, wherein the mAR agents enhance a cytotoxic activity of the one or more chemotherapeutic agents. In specific aspects of the method described herein the glioma is glioblastoma multiforme and the chemotherapeutic agent is temozolomide, salinomycin or a combination of temozolomide and salinomycin. In one aspect the one or more mAR activating agents or agonists selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene. In another aspect the mAR activating agent is selected from a testosterone or a dihydrotestosterone. In yet another aspect the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle. The conjugating agent used herein is a protein selected from a bovine serum albumin or a human serum albumin. The composition disclosed in the method of the present invention suppresses methylguanine methyltransferase (MGMT) activity, PI3K/Akt activity, extracellular signal regulated kinases (ERKs) or combinations thereof in one or more glial tumor cells. In a related aspect the mAR activating agent, agonists or both are administered orally, intravenously, intramuscularly, subcutaneously, intracranially or by any other suitable parenteral route. In other aspects the one or more chemotherapeutic agents and the mAR activating agents, agonists or both are administered in a dose ranging from 10 μM-10 mM and 1 nM-10 μM, respectively. In one aspect the mAR activating agents, agonists or both are administered prior to, concurrently or after the chemotherapy.

Another embodiment of the present invention relates to a composition for treating, ameliorating symptoms, delaying progression or combinations thereof of glioblastoma multiforme comprising: (i) temozolomide (TMZ), salinomycin or a combination of TMZ and salinomycin, (ii) a bovine serum albumin (BSA) conjugated testotsterone (BSA-T), a BSA conjugated dihydrotestosterone (BSA-DHT) or both, and (iii) one or more optional pharmaceutically acceptable excipients, wherein the composition simultaneously kills one or more glioblastoma multiforme cells and protects one or more brain cells, neurons or both. In one aspect the composition is administered orally, intravenously, intramuscularly, subcutaneously, intracranially or by any other suitable parenteral route. In another aspect the TMZ is administered in a dose ranging from 10 μM-10 mM. Salinomycin can be administered in a dose ranging from about 5 μM-10 mM. In yet another aspect the BSA-T, BSA-DHT or both are administered in a dose ranging from 1 nM-10 μM prior to, concurrently or after the administration of TMZ, salinomycin or a combination of TMZ and salinomycin.

In yet another embodiment the present invention discloses a method of treating, ameliorating symptoms, delaying progression or combinations thereof of glioblastoma multiforme in a subject comprising the steps of: identifying the subject in need of the treatment, amelioration of the symptoms, delaying progression or combinations thereof of the glioblastoma multiforme and administering a therapeutically effective amount of a pharmaceutical composition sufficient to treat or ameliorate the symptoms of the one or more cancers in the subject comprising: (i) temozolomide (TMZ), salinomycin or a combination of TMZ and salinomycin, (ii) a bovine serum albumin (BSA) conjugated testosterone (BSA-T), a BSA conjugated dihydrotestosteroneone (BSA-DI-IT) or both, and (iii) one or more optional pharmaceutically acceptable excipients, wherein the composition simultaneously kills one or more glioblastoma multiforme cells and protects one or more brain cells, neurons or both. In one aspect the composition is administered orally, intravenously, intramuscularly, subcutaneously, intracranially or by any other suitable parenteral route. In another aspect the TMZ is administered in a dose ranging from 10 μM-10 mM. Salinomycin can be administered in a dose ranging from about 5 μM-10 mM. In yet another aspect the BSA-T, BSA-DHT or both are administered in a dose ranging from 1 nM-10 μM. In a related aspect the BSA-T, BSA-DHT or both are administered prior to, concurrently or after the administration of TMZ, salinomycin or a combination of TMZ and salinomycin.

The instant invention also provides a method of enhancing efficacy of a chemotherapy or radiation therapy in a subject suffering from glioblastoma multiforme comprising the step of: administering a bovine serum albumin (BSA) conjugated testotsterone (BSA-T), a BSA conjugated dihydrotestosteroneone (BSA-DHT) or both, wherein the BSA-T, BSA-DHT or both agents enhance a cytotoxic activity of ionizing radiation, temozolomide (TMZ), salinomycin or a combination of TMZ and salinomycin against the glioblastoma multiforme by simultaneously killing the one or more glioblastoma multiforme cells and protecting one or more brain cells, neurons or both. In one aspect the BSA-T, BSA-DHT or both suppresses methylguanine methyltransferase (MGMT) activity, PI3K/Akt activity, extracellular signal regulated kinases (ERKs) or combinations thereof in the one or more glioblastoma multiforme cells. In another aspect the composition is administered orally, intravenously, intramuscularly, subcutaneously, intracranially or by any other suitable parenteral route. In yet another aspect the TMZ is administered in a dose ranging from 10 μM-10 mM, salinomycin can be administered in a dose ranging from about 5 μM-10 mM and the BSA-T, BSA-DHT or both are administered in a dose ranging from 1 nM-10 μM a related aspect the BSA-T, BSA-DHT or both are administered prior to, concurrently or after the administration of TMZ.

### BRIEF DESCRIPTION OF THE DRAWINGS

For a more complete understanding of the features and advantages of the present invention, reference is now made to the detailed description of the invention along with the accompanying figures and in which:

FIG. 1 shows the results of flow cytometric analysis of a putative mAR in A172 human glioblastoma cells;

FIGS. 2A and 2B show the effect of activating the mAR on ERK (FIG. 2A) and Akt (FIG, 2B) phosphorylation. DHT-BSA was used as the mAR ligand and applied for 30 min. Sham represents treatment with BSA alone, serving as our control. Bar graphs are densitometrtic representations of at least three Western blot runs;

FIG. 3 shows the activation of the mAR results enhances the cytotoxicity of Temozolamide (TMZ) in the A172 human glioblastoma cell line. A172 human glioblastoma cells were treated with either 2.5 mM or 5 mM TMZ in the presence or absence of increasing concentrations of the mAR-activating ligand, testosterone-BSA (TB). While 5 mM TMZ promoted a modest slight reduction in cell viability (as assessed by the level of calcein fluorescence—approx. 35%), the addition of both 1 uM or 5 uM of TB dramatically enhanced the degree of cytotoxicity;

FIG. 4 shows that the mAR ligand, Dihydrotestosterone-BSA (DHT-BSA) enhances the Temozolamide-induced increase in Caspase 3/7 activity. A172 human glioblastoma cells were used to evaluate the effects of the mAR agonist, DHT-BSA, on the effects of TMZ on caspase 3/7 activity, a marker of apoptotic cell death. DHT-BSA not only increased caspase 3/7 activity by itself, but also enhanced the effect of TMZ on caspase 3/7 activity; and

FIG. 5 shows that the mAR ligand, testosterone-BSA, protects hippocampal HT-22 cells from glutamate-induced cytotoxity. The neuronal HT-22 cell line was used to assess the effects of the BSA-conjugated testosterone, TBSA (a ligand of the mAR), on glutamate-induced cell death. While TBSA by itself had no effect on cell viability (as measured by the Calcein-am assay), it protected against glutamate-induced cytotoxicity.

In FIG. 6, A172 human glioblastoma cells were treated with increasing concentrations of the membrane impermeable androgen, TBSA (BSA-conjugated testosterone), which serves as our putative membrane androgen receptor activator. TBSA enhanced the sensitivity of the A172 glioblastoma cells to the cytotoxic effects of ionizing radiation (total dose of 5 Gy, administered at 6.3 Gy/min). Cell viability was measured using the Calcein-am assay following 6 hrs after the administration of the dose of ionizing radiation. The data are presented as a percentage of cell viability seen in the non-radiated (black bars) or radiated (checkered bars) in the absence of T-BSA.

FIGS. 7-12 relate to radiation sensitivity for temozolomide resistant glioblastoma cells. In Temozolomide-resistant T98g human glioblastoma cells, TBSA, at any of the concentrations tested (100 nM-40 microM), was ineffective at sensitizing the cells to ionizing radiation at a total dose of 5 Gy (at either 6 hr or 24 hr post-ionizing radiation treatment) (FIGS. 7-9). However, at 10 Gy (FIGS. 10-11), TBSA was effective at sensitizing these cells to ionizing radiation. At 20 Gy, the effect of TBSA was noted at concentrations as low as 1 microM (FIG. 12). Cell viability was measured using the Calcein-am assay following 6 and/or 24 hrs after the administration of the dose of ionizing radiation. The data are presented as a percentage of cell viability seen in the non-radiated (black bars) or radiated (checkered bars) in the absence of T-BSA.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “glioma” as used herein refers to a brain tumor that originates from glial cells, most often from astrocyte, such as glioblastoma multiforme and anaplastic astrocytoma, anaplastic oligodendroglioma and anaplastic oligoastrocytoma.

As used herein, the term “brain cell” refers to those cells that are found in, about or associated with cells of the central nervous system and the brain, including, the lower, mid and upper cortex, immune cells and support cells associated therewith. Brain cells include all types of neurons, e.g., afferent neurons, efferent neurons, and interneurons, whether pseudounipolar, bipolar, multipolar and the like. Cells in the brain include glial cells, astrocytes, Schwann cells, Purkinje cells, and the like, as will be known to the skilled artisan.

As used herein, the term “neuron” refers to a morphologic and functional unit of the brain, spinal column, and peripheral nerves. “Neurons” include, but are not limited to, a heterogeneous population of neuronal types having singular or multiple transmitters and/or singular or multiple functions; preferably, these are cholinergic and sensory neurons. As used herein, the phrase “cholinergic neuron” means neurons of the Central Nervous System (CNS) and Peripheral Nervous System (PNS) whose neurotransmitter is acetylcholine; exemplary are basal forebrain and spinal cord neurons.

As used herein, the term “chemotherapeutic agent” refers to chemical agents that preferentially kill neoplastic cells or disrupt the cell cycle of rapidly proliferating cells, used therapeutically to prevent or reduce the growth of neoplastic cells. Chemotherapeutic agents are also known as antineoplastic drugs or cytotoxic agents, and are well known in the art. Exemplary chemotherapeutic agents are vinca alkaloids, epipodophyllotoxins, anthracycline antibiotics, actinomycin D, salinomycin, plicamycin, puromycin, gramicidin D, paclitaxel (TAXOL®, Bristol Myers Squibb), colchicine, cytochalasin B, emetine, maytansine, and amsacrine (or “mAMSA”). The vinca alkaloid class is described in Goodman and Gilman's The Pharmacological Basis of Therapeutics, 1277-1280 (7th ed. 1985) (hereafter “Goodman and Gilman”). Exemplary of vinca alkaloids are vincristine, vinblastine, and vindesine. The epipodophyllotoxin class is described in Goodman and Gilman, supra at 1280-1281. Exemplary of epipodophyllotoxins are etoposide, etoposide orthoquinone, and teniposide. The anthracycline antibiotic class is described in Goodman and Gilman, supra at 1283-1285. Exemplary of anthracycline antibiotics are daunorubicin, doxorubicin, mitoxantraone, and bisanthrene. Actinomycin D, also called Dactinomycin, is described in Goodman and Gilman, supra at 1281-1283. Plicamycin, also called mithramycin, is described in Goodman and Gilman, supra at 1287-1288. Additional chemotherapeutic agents include cisplatin (PLATINOL®, Bristol Myers Squibb); carboplatin (PARAPLATIN®, Bristol Myers Squibb); mitomycin (MUTAMYCIN®, Bristol Myers Squibb); altretamine (HEXALEN®, U.S. Bioscience, Inc.); cyclophosphamide (CYTOXAN®, Bristol Myers Squibb); lomustine CCNU! (CEENU®, Bristol Myers Squibb); carmustine BCNU! (BICNU®, Bristol Myers Squibb). Methods of administering chemotherapeutic drugs vary depending upon the specific agent used, as would be known to one skilled in the art. Depending upon the agent used, chemotherapeutic agents may be administered, for example, by injection (intravenously, intramuscularly, intraperitoneally, subcutaneously, intratumor, intrapleural) or orally.

As used herein, “chemotherapy” includes treatment with a single chemotherapeutic agent or with a combination of agents. In a subject in need of treatment, chemotherapy may be combined with surgical treatment or radiation therapy, or with other antineoplastic treatment modalities.

The term “alkylating agent” as used herein includes, but is not limited to, alkyl sulfonates, aziridines, epoxides, ethylenimines, methylmelamines, nitrogen mustards, nitrosoureas, imidazotetrazinones, dacarbazine, mannomustine, mitobronitol, mitolactol, pipobroman and procarbazine.

As used herein the term “androgens” refer to steroids that develop and maintain primary and secondary male sex characteristics. Androgens are derivatives of cyclopentanoperhydrophenanthrene. Endogenous androgens are C-19 steroids with a side chain at C-17, and with two angular methyl groups. Testosterone is the primary endogenous androgen. Methyltestosterone is a synthetic derivative of testosterone suitable for oral administration. Androgens suitable for use in methods of the present invention include, e.g., testosterone, dihydrotestosterone, active metabolites of testosterone, and synthetic derivatives of testosterone such as testosterone propionate, testosterone cypionate, and fluoxymesterone.

The terms “testosterone”, “a testosterone” and the like are used interchangeably here and are intended to include the naturally occurring hormone known as testosterone having the chemical name 17-β-hydroxyandrost-4-en-3-one which may be isolated and purified from nature or synthetically produced in any manner. These terms are also intended to encompass the commonly occurring reduced version of testosterone having been reduced by 5 α-reductase to 5 α-dihydroxytestosterone which is also referred to here as dihydrotestosterone or simply “a testosterone.” A dihydrotestosterone may be isolated from nature but is preferably synthetically produced and purified. Testosterone USP is a white or creamy-white crystalline powder having a molecular weight of 288.43.

The term “testosterone derivative” refers to any androgen hormone for pharmaceutical use. The term includes testosterone esters, i.e. compounds where the “H” of the “OH” group is replaced with an alkyl group, e.g. propionate, cypionate and enanthate. Other pharmaceutically acceptable derivatives include methyltestosterone, methandrostenolone, fluovymesterone and danazol. A number of useful derivatives of testosterone are disclosed within the Physician's Desk Reference (most recent edition) as well as Harrison's Principles of Internal Medicine. In addition, applicants refer to U.S. Pat. No. 5,536,714 issued Jul. 16, 1996; U.S. Pat. No. 5,824,668 issued Oct. 20, 1998; U.S. Pat. No. 3,980,638 issued Sep. 14, 1996; U.S. Pat. No. 4,031,117 issued Jun. 21, 1977; U.S. Pat. No. 4,085,202 issued Apr. 18, 1978; U.S. Pat. No. 4,197,286 issued Apr. 8, 1980; 4,507,290 issued Mar. 26, 1985 and U.S. Pat. No. 5,622,944 issued Apr. 22, 1997 all of which are incorporated herein by reference to disclose and describe testosterone derivatives and formulations.

The term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

The term “androgen receptor ” or “AR” refers to the androgen receptor protein as defined by its conserved amino acid coding sequence in an active or native structural conformation. Nucleic acid sequences encoding androgen receptors have been cloned and sequenced from numerous organisms. Representative organisms and GenBank® accession numbers for androgen receptor sequences therefrom include the following: frog (Xenopus laevis; U67129), mouse (Mus musculus, 109558), rat (Rattus norvegicus, 292896), human (Homo sapiens, 105325), rabbit (Oryctolagus cuniculus, 577829), cow (Bos taurus, 275313, Z75314, Z75315), canary (Serinus canaria, 414734), whiptail lizard (Cnemidophous uniparens, 1195596), and canine (Canis familiaris, AF197950). It must be noted that the membrane androgen receptor (mAR) as described in various embodiments of the present invention has not been cloned. The present inventors have previously characterized the mAR's and have found them to be pharmacologically and functionally distinct from the classical AR described hereinabove.

The terms “activating agent”, “agonist” and “agonistic” when used herein refer to a molecule which is capable of, directly or indirectly, substantially inducing, promoting or enhancing biological activity or activation of a molecule such as the androgen receptor (AR).

The term “pharmaceutically acceptable” refers to the carrier, diluent or excipient and must be compatible with the other ingredients of the formulation and not deleterious to the recipient thereof.

The terms “administration of” or “administering a” compound should be understood as providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The membrane androgen receptor-specific binding agents, activating agents or agonists may be contacted to cells, in vitro or in vivo, in a variety of dosage forms. For example, the membrane androgen receptor-specific binding agents may be provided to a patient through a variety of locations, e.g., oral, intravenous (bolus or infusion), intraperitoneal, subcutaneous, intramuscular, pulmonary, intradural, intrarenal, percutaneous, and the like in a form adapted for such delivery as is well known to those of ordinary skill in the pharmaceutical arts.

Dosage forms: A dosage unit for use of the membrane androgen receptor-specific binding/activating agents or agonists of the present invention may be a single compound or mixtures thereof. For example, the agent may be included with other compounds such as a potentiator or counter-activator (e.g., an antagonist of the intracellular androgen receptor). The compounds may be mixed together, form ionic or even covalent bonds. The membrane androgen receptor-specific binding agents of the present invention may be administered in oral, intravenous (bolus or infusion), intraperitoneal, subcutaneous, intrapulmonary, intramuscular form, and the like, using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the membrane androgen receptor-specific binding agents of the present invention to a patient in need of therapy that includes, alone in combination, an agent that causes: intracellular androgen receptor activation, intracellular androgen receptor inactivation, membrane androgen receptor activation, membrane androgen receptor inactivation and combinations thereof.

The membrane androgen receptor-specific binding binding/activating agents or agonists may also be administered as any one of known salt forms. Membrane androgen receptor-specific binding agents are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the membrane androgen receptor-specific binding agents may be formulated to provide, e.g., maximum and/or consistent dosing for the particular form for oral, rectal, topical, intravenous injection or parenteral administration. While the membrane androgen receptor-specific binding agents may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions incorporated herein by reference.

For example, the membrane androgen receptor-specific binding/activating agents or agonists may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

Membrane androgen receptor-specific binding/activating agents or agonists may also be administered in the form of liposome delivery systems, e.g., small unilamellar vesicles, large unilamellar vesicles, and multilamellar vesicles, whether charged or uncharged. Liposomes may include one or more: phospholipids (e.g., cholesterol), stearylamine and/or phosphatidylcholines, mixtures thereof, and the like. Membrane androgen receptor-specific binding agents may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the membrane androgen receptor-specific binding agents may be coupled one or more biodegradable polymers to achieve controlled release of the membrane androgen receptor-specific binding agents, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polyeyanoacylates, and crosslinked or amphipathic block copolymers of hydrogels, mixtures thereof, and the like.

A capsule or gelatin capsules (gelcaps) may be loaded with the membrane androgen receptor-specific binding/activating agents or agonists and one or more powdered carriers or fillers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference,

For direct delivery to the nasal passages, sinuses, mouth, throat, esophagus, trachea, lungs and alveoli, the membrane androgen receptor-specific binding/activating agents or agonists may also be delivered as an intranasal form via use of a suitable intranasal vehicle. Generally, the smaller the particle the deeper the delivery, as such, the membrane androgen receptor-specific binding agents may be prepared into nanoparticles by, e.g., freeze-spraying, to form individual nanoparticles. For dermal and transdermal delivery, the membrane androgen receptor-specific binding agents may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of membrane androgen receptor-specific binding agents may include the following forms.

Capsules: Capsules may be prepared by filling standard two-piece hard gelatin capsules each with. e.g., 10 to 500 milligrams of powdered active ingredient (e.g., membrane androgen receptor-specific binding/activating agent(s) or agonists), 5 to 150 milligrams of lactose, 5 to 50 milligrams of cellulose and 6 milligrams magnesium stearate.

Soft Gelatin Capsules: A mixture of active ingredient is dissolved in a digestible oil such as soybean oil, cottonseed oil or olive oil. For example, the active ingredient is prepared and injected by using a positive displacement pump into gelatin to form soft gelatin capsules containing, e.g., 1-500 milligrams of the membrane androgen receptor-specific binding/activating agents or agonists. The capsules are washed and dried.

Tablets: A large number of tablets are prepared by conventional procedures so that the dosage unit was, e.g., 10-500 milligrams of membrane androgen receptor-specific binding/activating agents or agonists, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution: A parenteral composition suitable for administration by injection is prepared by, e.g., stirring 0.1 to 1.5% by weight of membrane androgen receptor-specific binding/activating agents or agonists in deionized water (or other solvent) and mixed with, e.g., up to 10% by volume propylene glycol and water. The solution is made isotonic with sodium chloride and sterilized using, e.g., ultrafiltration.

Suspension: An aqueous suspension is prepared for oral administration so that each 5 include, e.g., 1-500 mg of the membrane androgen receptor-specific binding/activating agents or agonists, 200 mg of sodium carboxymethyl cellulose, 5 mg of sodium benzoate, 1.0 g of sorbitol solution, U.S.P., and 0.025 ml of vanillin. For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

The terms “effective amount” or “therapeutically effective amount” refers to the amount of the subject compound that will elicit the biological or medical response of a tissue, system, animal or human that is being sought by the researcher, veterinarian, medical doctor or other clinician. Such dosing amounts are readily ascertainable by those skilled in the art. For example, TMZ can be administered in doses ranging from 5 mg-240 mg and an initial TMZ treatment is usually about 75 mg/m² (e.g., TMZ treatment accompanied by radiation therapy). A standard maintenance dosing schedule (TMZ alone) can be between 150 mg-200 mg/m².

As used herein, the term “treatment” or “treating” includes any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The present invention teaches a method for enhancing the effectiveness of one or more chemotherapeutic agents and/or ionizing radiation (radiation therapy) directed against glial tumors. The present invention describes a novel target for androgens, the membrane androgen receptor (herein referred to as mAR), that when activated renders the tumor cell more vulnerable to the death promoting effects of the chemotherapeutic agent and/or ionizing radiation. The inventors further report that this same mechanism that promotes cell death in the glial tumor cells appears to promote the survival/viability of neuronal cells.

The present invention provides a number of significant advantages: (i) the invention relates to glial tumors, which are not addressed in other existing technologies (which principally address prostate cancer) and (ii) the present invention not only defines a means of destroying glial-based cancers, but also simultaneously affords protection of neurons, ensuring a more cell-targeted means of treating cancer.

Glioblastoma multiforme (GBM) is a grade IV astrocytoma that has the worst prognosis among brain cancers and currently is believed to occur at random with no known cause (Grossman and Batara, 2004; Yin et al., 2007). The only known risk factors are age, gender, and race, such that individuals who are 65 yrs and older, male, and Caucasian are at greatest risk for the disease (Curran et al., 1993; Wrensch et al., 2002; Chakrabarti et al., 2005). Generally, patients diagnosed with GBM have a median life expectancy of about one year, with only 42% surviving to six months and less than 5% surviving past two years (Ohgaki et al., 2004). Unfortunately, despite efforts to improve the primary modes of treatment (that include surgery (when possible), radiotherapy and chemotherapy), the median survival rate has hardly changed in the past 40 years (Mason and Cairncross, 2005). At present, the most commonly used chemotherapeutic for glioblastoma is the alkylating agent, Temozolomide (TMZ) (Yoshino et al., 2010). While TMZ has shown promise, its efficacy is often compromised due to the existence of enhanced DNA repair mechanisms (Friedman et al., 2000; Maxwell et al., 2008; Augustine et al., 2009) and/or enhanced anti-apoptotic mechanisms (Minniti et al., 2009). Of note, O6 methylguanine methyltransferase (MGMT), which removes the O6 methylguanine adducts introduced by TMZ (Kaina et al., 2007), has been found to be elevated in glioblastomas that are resistant to TMZ (Zhang et al., 2010). Further, studies have linked an enhanced expression/activity of the survival-promoting PI3K/Akt signaling pathway to the resistance of glioblastoma to standard therapies (Chakravarti et al., 2004; Cheng et al., 2009). As such, defining means of reducing MGMT and Akt activity and/or expression would be desirable.

While the expression of the classical androgen receptor (AR) has been described in astrocytomas, including glioblastoma, little is known about its role in the development/progression of glioblastoma. While the reported sex difference in incidence of glioblastoma may suggest an involvement of steroid hormones, no studies have effectively addressed the role of androgens and/or their receptors in glioblastoma. The present inventors have recently characterized a novel putative membrane androgen receptor (termed mAR) in C6 glioma cells, which when activated, greatly enhances the sensitivity of these cells to a cytotoxic insult (Gatson et al., 2006; Gatson and Singh, 2007). In these same cells, activation of the mAR also resulted in a suppression of ERK and Akt phosphorylation (Gatson et al., 2006), a desired effect in the treatment of glioblastoma, In fact, these two cell signaling effectors are being considered as relevant targets in the development of new therapeutic strategies for glioblastoma (Clarke et al., 2010). The inventors expanded the analysis to include preliminary studies using the human glioblastoma cell line, A 172, and found that activation of the mAR sensitizes the A172 cells to the toxic effects of TMZ. However, activation of the mAR by itself (i.e., without accompanying cytotoxic insult) did not cause cell death, an effect deemed by the inventors as desirable in the context of glioblastoma treatment since healthy (non cancerous) glial cells that express the mAR are not destroyed in the face of androgen exposure.

Androgens, androgen receptors and glioblastoma: Androgens, like testosterone and dihydrotestosterone, are conventionally believed to exert their effects through activation of the “classical” androgen receptors. As members of the nuclear receptor superfamily, these receptors are transcriptional regulators and mediate the so-called “genomic” effects of androgens (Mangelsdorf et al., 1995). More recently, however, these intracellular receptors have also been implicated in regulating such cellular processes as cell signaling. In addition to the classical AR, a putative membrane-associated androgen receptor (termed here as mAR), has been recently characterized and implicated in “non-genomic” effects that influence cellular growth, cell signaling, and survival (Braun and Thomas, 2004; Hatzoglou et al., 2005; Gatson et al., 2006). While membrane androgen receptors have been reported in various tissues (including kidney, liver and prostate), the inventors were the first to describe the mAR in both normal astrocytes and in glial tumor cells ((Gatson et al., 2006; Gatson and Singh, 2007). In these cells, activation of the mAR led to an increased vulnerability of the glial cells to a cytotoxic insult, and was accompanied by suppression of the ERK1/2 and PI3K/Akt signaling pathways. It is worth noting that others have shown the benefit of activating the mAR in other cancer cell types, including breast (T47D) and prostate cancer (LnCaP and DU145) lines (Kampa et al., 2006; Papadopoulou et al., 2008).

Akt and cancer: A key effector of the signaling pathway initiated by phosphoinositide (PI)-3 kinase (PI3K) is the PKA- and PKC-related signaling protein, Akt (also known as PKB) (Franke et al., 1997). Activation of this signaling protein is implicated in a number of cellular processes. Of particular interest is its involvement in the inhibition of apoptosis (Dudek et al., 1997). The dysregulation of this protein has been shown to play an important role in the formation and proliferation of gliomas among other cancers (Sonoda et al., 2001; Jacques-Silva et al., 2004; Fujiwara et al., 2007). In glioblastoma, Akt has been shown to be upregulated, and in fact, the higher phosphorylated status of Akt is associated with a worse prognosis. Inhibition of this pathway, however, has been shown in several studies to slow the growth of glioma cells (Suzuki et al., 2010; Holland et al., 2000; Rajasekhar et al., 2003). These findings and the prominent occurrence of upregulation of this protein in glioblastoma support the direction of targeting Akt as a potentially viable avenue for therapy in this devastating disease (Akhavan et al., 2010).

The findings of the present invention indicate that a novel membrane androgen receptor (mAR) serves as a new and innovative therapeutic target for the treatment of glioblastoma. Specifically, activating the mAR sensitizes glioblastoma cells to the cytotoxic effects of TMZ, salinomycin or a combination of TMZ and salinomycin and further, renders TMZ-insensitive and/or salinomycin-insensitive tumors responsive to treatment, through the mechanisms described herein below. The present inventors conducted a systematic analysis of the expression and function of the mAR in two human glioblastoma cell lines (with differential sensitivity to TMZ) and “normal” human astrocytes, to not only understand androgen glia-biology, but also to discover an unique and unexplored therapeutic avenue for glioblastoma (i.e., targeting a membrane associated androgen receptor).

A commonly used method for targeting membrane steroid hormone receptors has been achieved by conjugation of the steroid to a macromolecule, such as bovine serum albumin (BSA) (Erlanger et al., 1957; Zheng et al., 1996). The principle underlying its utility is that the ‘bulky’ BSA-conjugated steroids cannot enter into the cells due to their large size, but they are allowed to interact with their cognate membrane receptors. As such, any effect of these conjugates is interpreted as an effect of activating a plasma membrane-associated receptor. Indeed, several steroid-BSA conjugates are commercially available and frequently used by different groups to explore the biology of these membrane steroid hormone receptors.

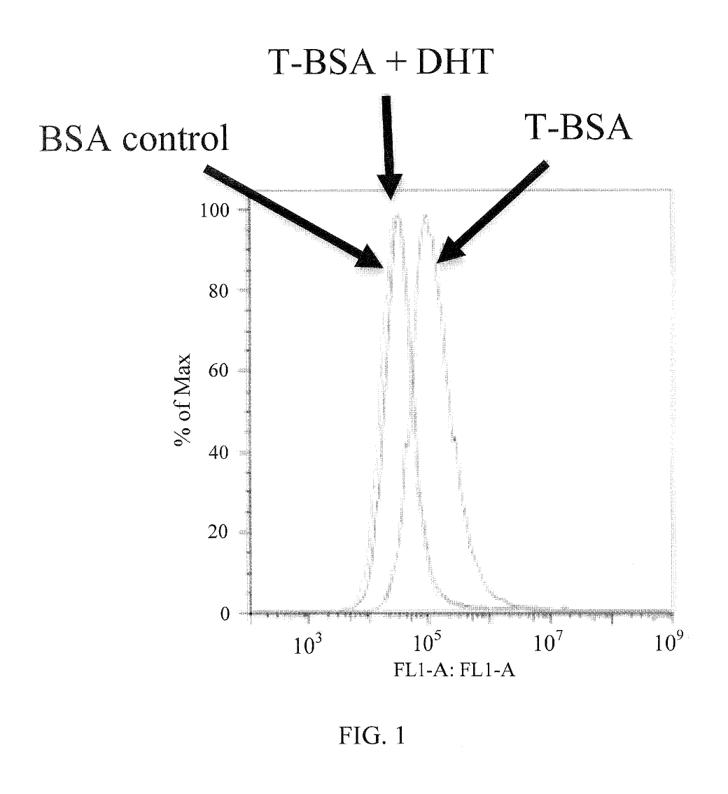

FIG. 1 shows the results of flow cytometric analysis of a putative mAR in A172 human glioblastoma cells and a measurement of androgen (Dihydrotestosterone, DHT) displaceable binding sites on the surface of A172 cells. The population histograms depict the frequency distribution of the cells labeled with a fluorescently tagged, BSA-conjugated testosterone (Testosterone-BSA-FITC, abbreviated as T-BSA), or T-BSA in the presence of unlabeled DHT, serving as the “displacer”. The data reveal a clear leftward shift of fluorescence intensity, relative to the T-BSA incubated cells, supporting the existence of DHT-displaceable binding sites on the cell surface. As a point of reference, binding of the negative control, BSA alone conjugated to FITC (“BSA control”, serving as an index of non-specific binding), resulted in a profile similar to the DHT-displaced group (T-BSA+DHT) to the extent that the two population histograms are nearly perfectly overlapping. Also note that the x-axis is on a log scale.

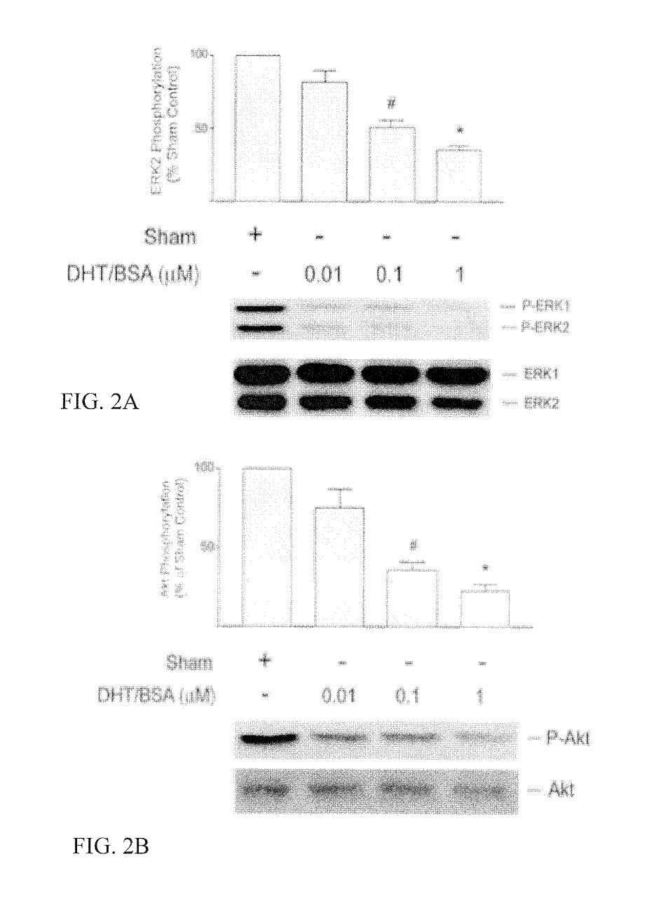

FIGS. 2A and 2B show the effect of activating the mAR on ERK (FIG. 2A) and Akt (FIG. 2B) phosphorylation. DHT-BSA was used as the mAR ligand and applied for 30 min. Sham represents treatment with BSA alone, serving as our control. Bar graphs are densitometrtic representations of at least three Western blot runs. FIGS. 2A and 2B demonstrate that activation of the putative mAR results in a concentration-dependent reduction in the phosphorylation of ERK and Akt in C6 glioma cells, desirable effects when considering the promotion of cell death or the reduction in cell proliferation.

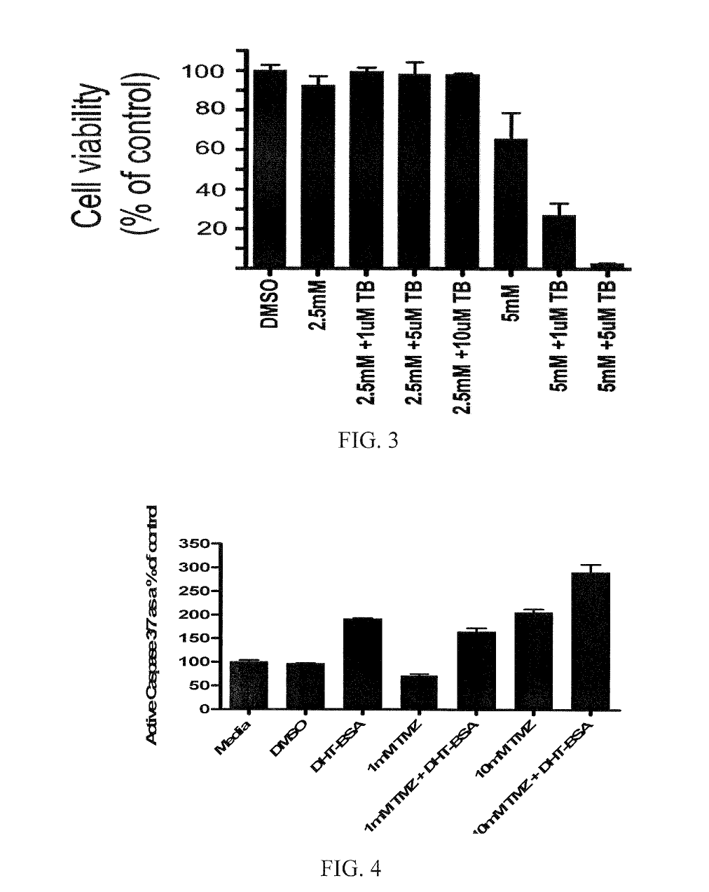

FIG. 3 shows the activation of the mAR results enhances the cytotoxicity of Temozolamide (TMZ) in the A172 human glioblastoma cell line. A172 human glioblastoma cells were treated with either 2.5 mM or 5 mM TMZ in the presence or absence of increasing concentrations of the mAR-activating ligand, testosterone-BSA (TB). While 5 mM TMZ promoted a modest slight reduction in cell viability (as assessed by the level of calcein fluorescence—approx. 35%), the addition of both 1 uM or 5 uM of TB dramatically enhanced the degree of cytotoxicity. The data in FIG. 3 supports the hypothesis that activation of the mAR enhances the cytotoxicity of Temozolamide (TMZ) in the A172 human glioblastoma cell line.

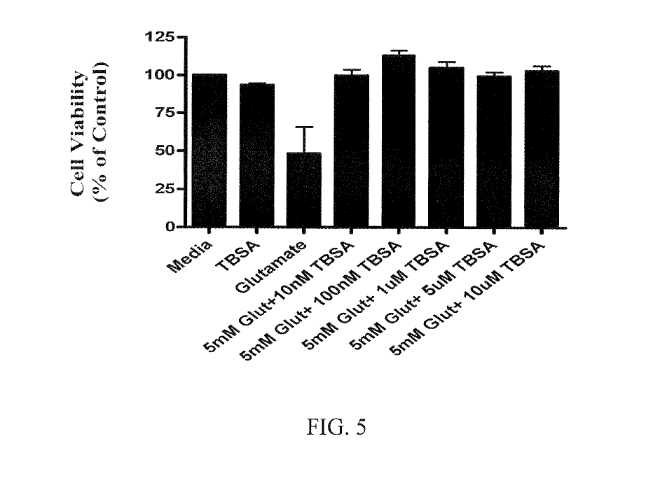

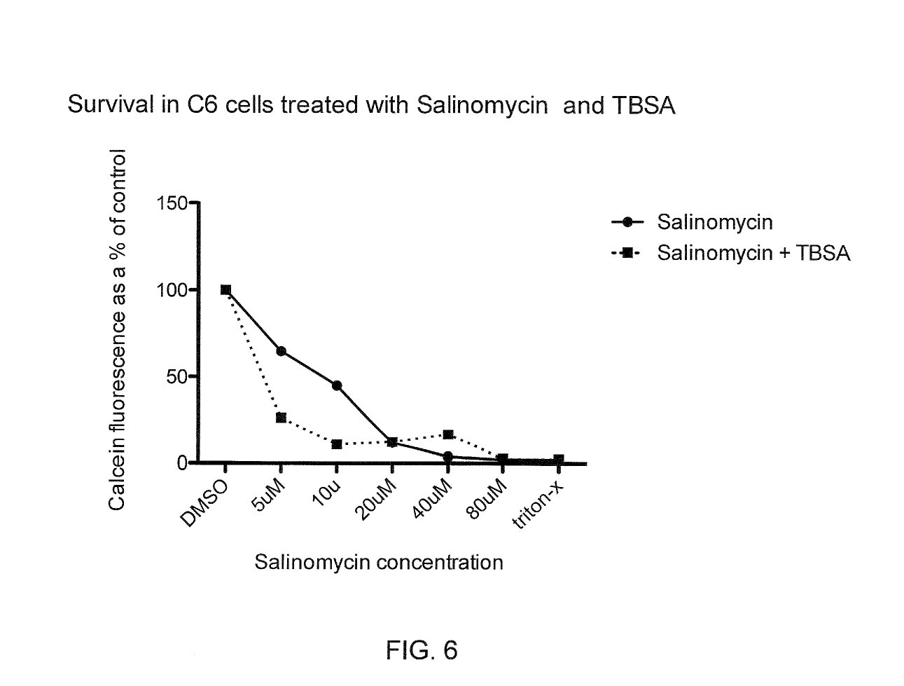

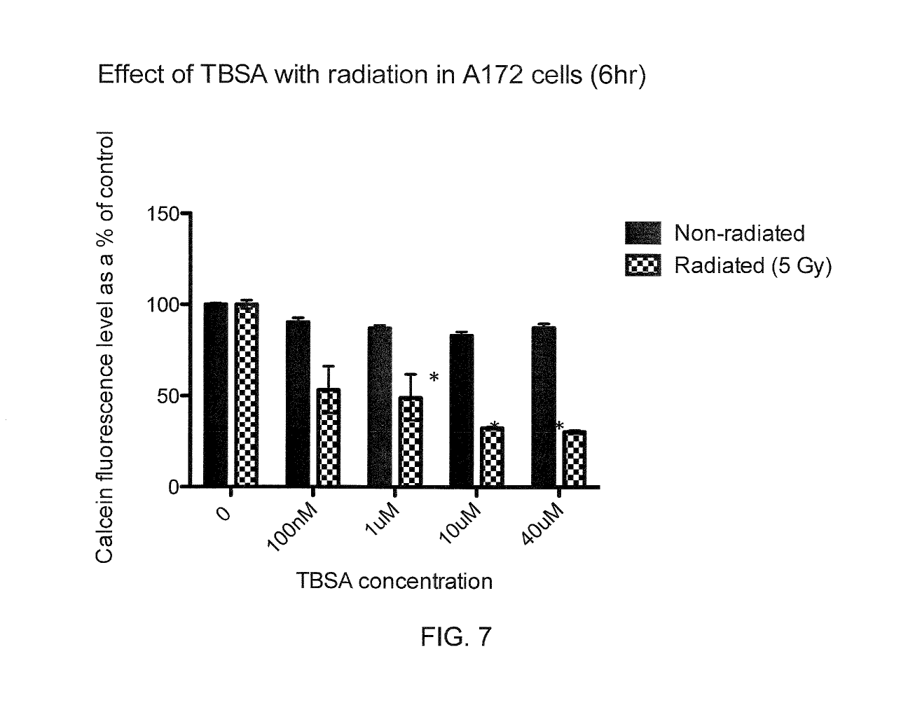

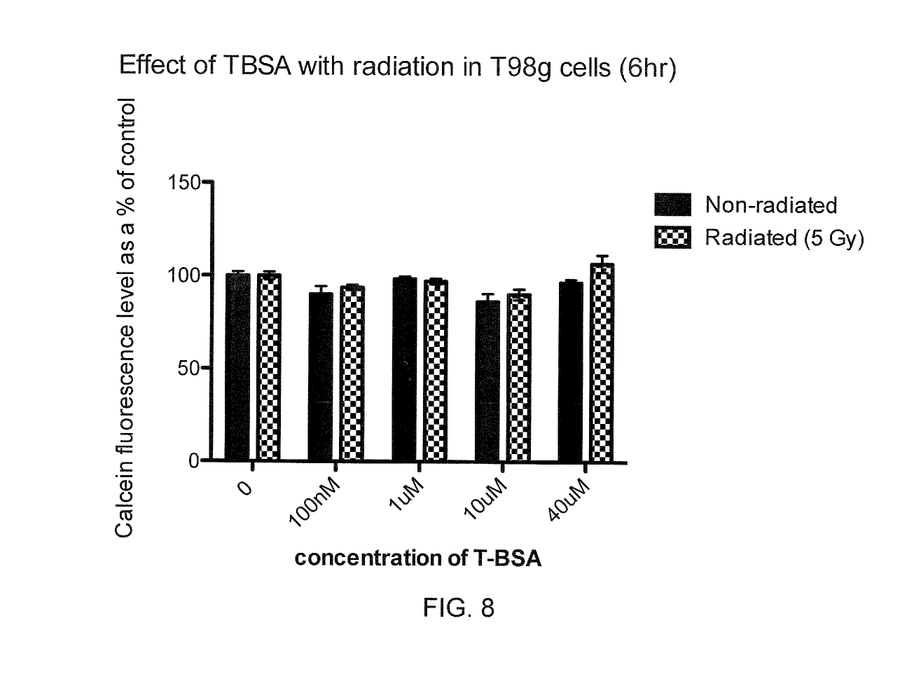

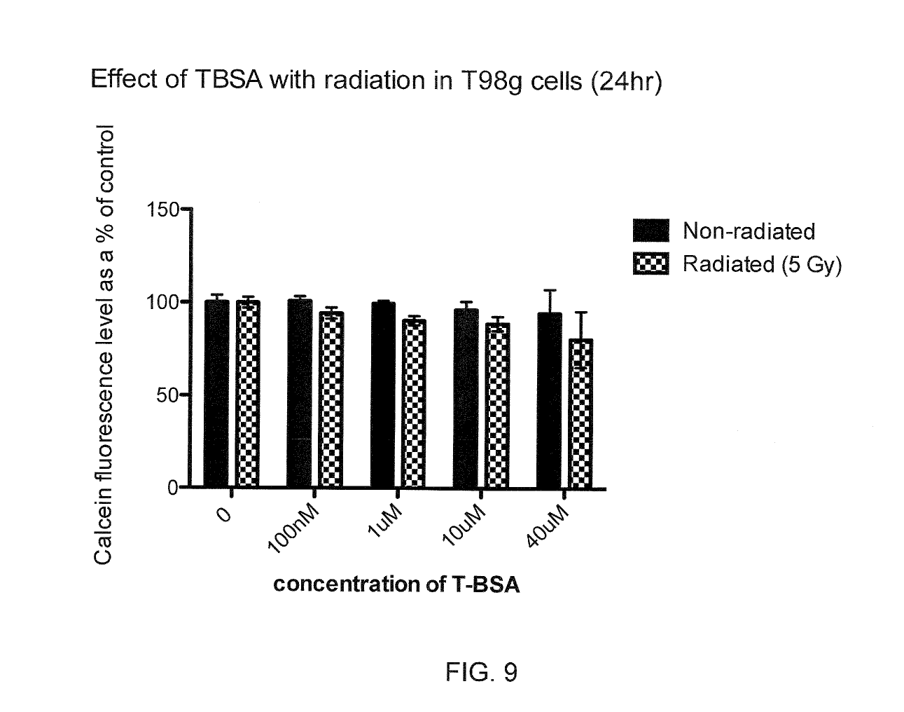

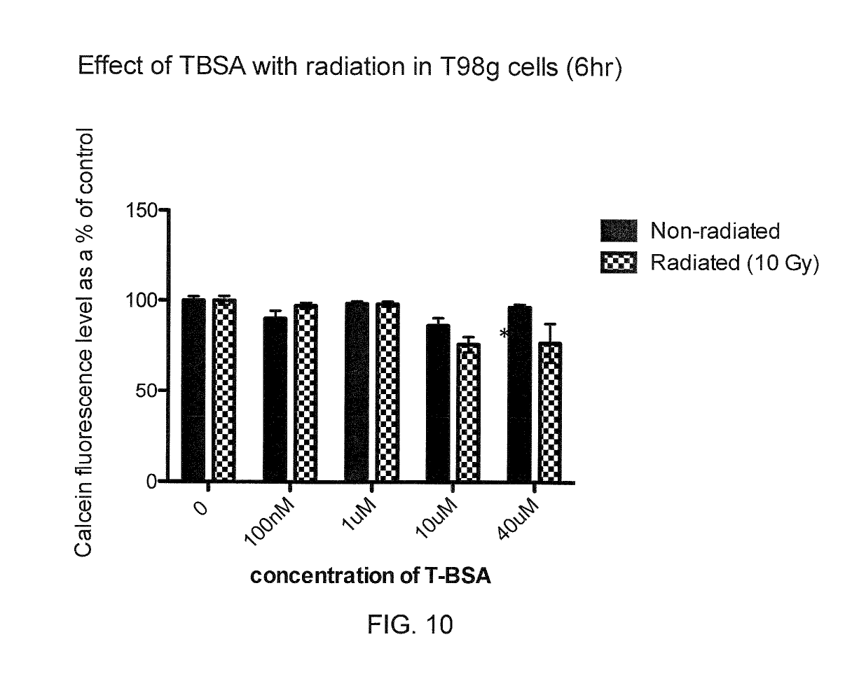

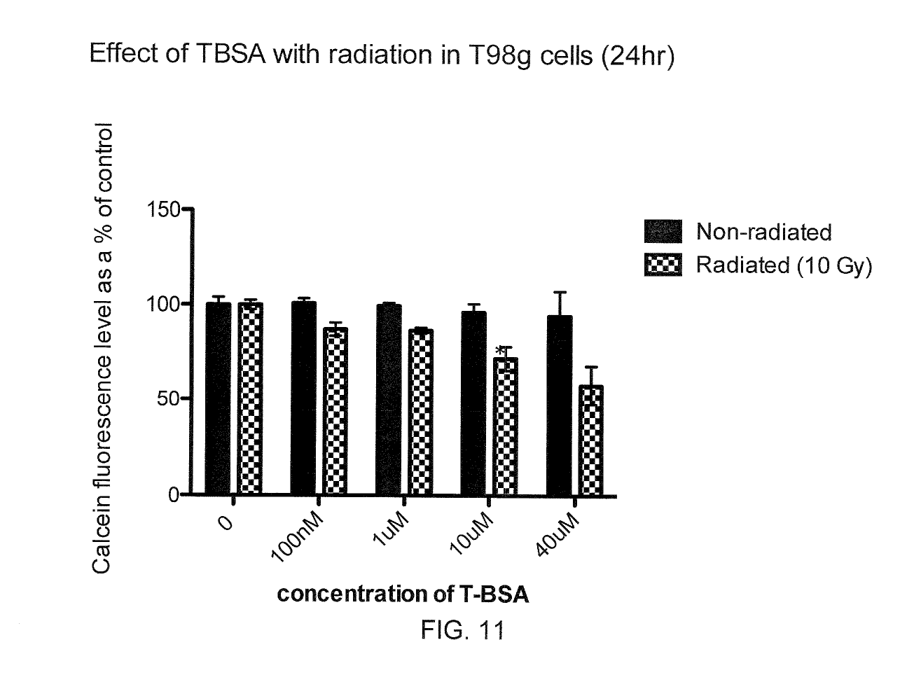

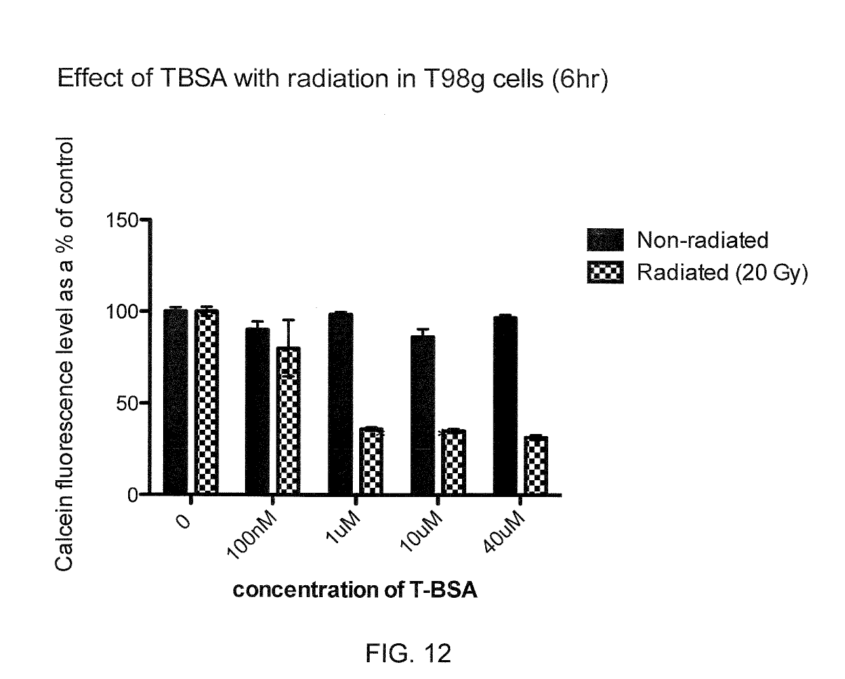

From FIG. 4 it can be seen that that the Dihydrotestosterone-BSA (DHT-BSA) enhances the Temozolamide-induced increase in Caspase 3/7 activity. This was evaluated using A172 human glioblastoma cells. Caspase 3/7 activity is a marker of apoptotic cell death. DHT-BSA not only increased caspase 3/7 activity by itself, but also enhanced the effect of TMZ on caspase 3/7 activity. In FIG. 5 testosterone-BSA (T-BSA) protects hippocampal HT-22 cells from glutamate-induced cytotoxity. The neuronal HT-22 cell line was used to assess the effects of the BSA-conjugated testosterone, TBSA (a ligand of the mAR), on glutamate-induced cell death. While TBSA by itself had no effect on cell viability (as measured by the Calcein-am assay), it protected against glutamate-induced cytotoxicity. The data collectively support the claim that activation of the mAR sensitizes/enhances the glial tumor cell to the toxic consequences of TMZ, while protecting neurons.

FIG. 5 demonstrates the effects of treating glioma cells with salinomycin in combination with TBSA. C6 glioma cells were treated with increasing concentrations of the cancer therapeutic, salinamycin, a drug with a high profile of selectivity for cancer stem cells. 10 microM of the membrane impermeable androgen, TBSA (BSA-conjugated testosterone), which serves as a membrane androgen receptor activator, increased the sensitivity of the cells to salinamycin as evidenced by a leftward shift of the concentration response. Cell viability was measured using the Calcein-am assay following 48 hrs after the administration of TBSA and salinamycin. The data are presented as a percentage of cell viability seen in non-TBSA/salinamycin treated cells. The group labeled as Triton-X serves as our cell death-inducing control.

In FIG. 6, A172 human glioblastoma cells were treated with increasing concentrations of the membrane impermeable androgen, TBSA (BSA-conjugated testosterone), which serves as our putative membrane androgen receptor activator. TBSA enhanced the sensitivity of the A172 glioblastoma cells to the cytotoxic effects of ionizing radiation (total dose of 5 Gy, administered at 6.3 Gy/min). Cell viability was measured using the Calcein-am assay following 6 hrs after the administration of the dose of ionizing radiation. The data are presented as a percentage of cell viability seen in the non-radiated (black bars) or radiated (checkered bars) in the absence of T-BSA.

FIGS. 7-12 relate to radiation sensitivity for temozolomide resistant glioblastoma cells. In Temozolomide-resistant T98g human glioblastoma cells, TBSA, at any of the concentrations tested (100 nM-40 microM), was ineffective at sensitizing the cells to ionizing radiation at a total dose of 5 Gy (at either 6 hr or 24 hr post-ionizing radiation treatment) (FIGS. 7-9). However, at 10 Gy (FIGS. 10-11), TBSA was effective at sensitizing these cells to ionizing radiation. At 20 Gy, the effect of TBSA was noted at concentrations as low as 1 microM (FIG. 12). Cell viability was measured using the Calcein-am assay following 6 and/or 24 hrs after the administration of the dose of ionizing radiation. The data are presented as a percentage of cell viability seen in the non-radiated (black bars) or radiated (checkered bars) in the absence of T-BSA.

Thus, the following non-limiting embodiments are also provided:
1. A composition for enhancing simultaneously the effectiveness of one or more chemotherapeutic agents and for protecting one or more brain cells, neurons or both, wherein the chemotherapeutic agents treat, ameliorate symptoms, or delay a progression of one or more gliomas comprising:
   one or more chemotherapeutic agents selected from the group consisting of dacarbazine alkylating agents, salinomycin, temozolomide, procarbazine, nitrosoureas, bis-chloronitrosourea, lomustine, and platinum based chemotherapeutic agents;
   one or more membrane androgen receptor (mAR) activating agents, agonists or both, wherein the agents are selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene; and one or more optional pharmaceutically acceptable excipients.
2. The composition of embodiment 1, wherein the one or more gliomas are selected from the group consisting of astrocytomas, ependymal tumors, glioblastoma multiforme, and primitive neuroectodermal tumors.
3. The composition of embodiment 1, wherein the mAR activating agent is selected from a testosterone or a dihydrotestosterone.
4. The composition of embodiment 1, wherein the synthetic derivatives of testosterone comprise testosterone propionate, testosterone cypionate, and fluoxymesterone.
5. The composition of embodiment 1, wherein the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle.
6. The composition of embodiment 5, wherein the conjugating agent is a protein selected from a bovine serum albumin or a human serum albumin.
7. The composition of embodiment 1, wherein the composition is administered orally, intravenously, intramuscularly, subcutaneously, intracranially or by another suitable parenteral route.
8. The composition of embodiment 1, wherein the one or more chemotherapeutic agents are administered in a dose ranging from 10 μM-10 mM.
9. The composition of embodiment 1, wherein the one or more mAR activating agents or agonists are administered in a dose ranging from 1 nM-10 μM.
10. A method of treating, ameliorating symptoms, delaying progression or combinations thereof of one or more glial cancers in subject comprising the steps of:
   identifying the subject in need of the treatment, amelioration of the symptoms, delaying the progression or combinations thereof of the glial cancers; and
   administering a therapeutically effective amount of a pharmaceutical composition sufficient to treat, ameliorate symptoms, delay progression or combinations thereof of the one or more cancers in the subject comprising:
   one or more chemotherapeutic agents, wherein the one or more chemotherapeutic agents are selected from the group consisting of dacarbazine alkylating agents, salinomycin, temozolomide, procarbazine, nitrosoureas, bis-chloronitrosourea, lomustine, and platinum based chemotherapeutic agents;
   one or more membrane androgen receptor (mAR) activating agents, agonists or both wherein the agents are selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene; and one or more optional pharmaceutically acceptable excipients, wherein the composition simultaneously kills one or more glial cancer cells and protects one or more brain cells, neurons or both.
11. The method of embodiment 10, wherein the glial cancers are selected from the group consisting of astrocytomas, ependymal tumors, glioblastoma multiforme, and primitive neuroectodermal tumors.
12. The method of embodiment 10, wherein the mAR activating agent, agonist or both are selected from a testosterone or a dihydrotestosterone.
13. The method of embodiment 10, wherein the synthetic derivatives of testosterone comprise testosterone propionate, testosterone cypionate, and fluoxymesterone.
14. The method of embodiment 10, wherein the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle.
15. The method of embodiment 10, wherein the conjugating agent is a protein selected from a bovine serum albumin or a human serum albumin.
16. The method of embodiment 10, wherein the composition is administered orally, intravenously, intramuscularly, subcutaneously, intracranially or another suitable parenteral route.
17. The method of embodiment 10, wherein the one or more chemotherapeutic agents are administered in a dose ranging from 10 μM-10 mM.
18. The method of embodiment 10, wherein the one or more mAR activating agents or agonists are administered in a dose ranging from 1 nM-10 μM.
19. A therapeutic composition comprising one or more membrane androgen receptor (mAR) activating agents, agonists or both in an amount sufficient to enhance a cytotoxic activity of one or more chemotherapeutic agents against one or more glioma cells by a suppression of one or more cell signaling effectors, pathways or both, wherein the composition simultaneously kills the one or more glioma cells and protects one or more brain cells, neurons or both.
20. The composition of embodiment 19, wherein the chemotherapeutic agent is temozolomide or salinomycin.
21. The composition of embodiment 19, wherein the one or more mAR activating agents or agonists selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene.
22. The composition of embodiment 19, wherein the mAR activating agent is selected from a testosterone or a dihydrotestosterone.
23. The composition of embodiment 19, wherein the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle.
24. The composition of embodiment 23, wherein the conjugating agent is a protein selected from a bovine serum albumin or a human serum albumin.
25. The composition of embodiment 19, wherein the composition suppresses methylguanine methyltransferase (MGMT) activity, PI3K/Akt activity, extracellular signal regulated kinases (ERKs) or combinations thereof in the one or more glioma cells.
26. A method for enhancing simultaneously the efficacy of a chemotherapy and for protecting one or more brain cells, neurons or both in a subject comprising the steps of:
   identifying the subject suspected of having a need for the treatment of a glioma;
   administering one or more membrane androgen receptor (mAR) activating agents, agonists or both, wherein the mAR agents enhance a cytotoxic activity of the one or more chemotherapeutic agents.
27. The method of embodiment 26, wherein the glioma is glioblastoma multiforme.
28. The method of embodiment 26, wherein the chemotherapeutic agent is temozolomide or salinomycin.
29. The method of embodiment 026, wherein the one or more mAR activating agents or agonists selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene.
30. The method of embodiment 26, wherein the mAR activating agent is selected from a testosterone or a dihydrotestosterone.
31. The method of embodiment 26, wherein the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle.
32. The composition of embodiment 31, wherein the conjugating agent is a protein selected from a bovine serum albumin or a human serum albumin.
33. The method of embodiment 26, wherein the composition suppresses methylguanine methyltransferase (MGMT) activity, PI3K/Akt activity, extracellular signal regulated kinases (ERKs) or combinations thereof in one or more glial tumor cells.
34. The method of embodiment 26, wherein the mAR activating agent, agonists or both are administered orally, intravenously, intramuscularly, subcutaneously, intracranially or by another suitable parenteral route.
35. The method of embodiment 26, wherein the one or more chemotherapeutic agents are administered in a dose ranging from 10 μM-10 mM.
36. The method of embodiment 26, wherein the mAR activating agents, agonists or both are administered in a dose ranging from 1 nM-10 μM.
37. The method of embodiment 26, wherein the mAR activating agents, agonists or both are administered prior to, concurrently or after the chemotherapy.
38. A composition for treating, ameliorating symptoms, delaying progression or combinations thereof of glioblastoma multiforme comprising:
   temozolomide (TMZ), salinomycin or a combination of TMZ and salinomycin;
   a bovine serum albumin (BSA) conjugated testotsterone (BSA-T), a BSA conjugated dihydrotestosteroneone (BSA-DHT) or both; and
   one or more optional pharmaceutically acceptable excipients, wherein the composition simultaneously kills one or more glioblastoma multiforme cells and protects one or more brain cells, neurons or both.
39. The composition of embodiment 38, wherein the composition is administered orally, intravenously, intramuscularly, subcutaneously, intracranially or by another suitable parenteral route.
40. The composition of embodiment 38, wherein the TMZ is administered in a dose ranging from 10 μM-10 mM and salinomycin is administered in a dose ranging from about 5 μM-10 mM.
41. The composition of embodiment 38, wherein the BSA-T, BSA-DHT or both are administered in a dose ranging from 1 nM-10 μM.
42. The composition of embodiment 38, wherein the BSA-T, BSA-DHT or both are administered prior to, concurrently or after the administration of TMZ, salinomycin or a combination of TMZ and salinomycin.
43. A method of treating, ameliorating symptoms, delaying progression or combinations thereof of glioblastoma multiforme in a subject comprising the steps of:
   identifying the subject in need of the treatment, amelioration of the symptoms, delaying progression or combinations thereof of the glioblastoma multiforme; and
   administering a therapeutically effective amount of a pharmaceutical composition sufficient to treat or ameliorate the symptoms of the one or more cancers in the subject comprising:
      temozolomide (TMZ), salinomycin or a combination of TMZ and salinomycin; a bovine serum albumin (BSA) conjugated testosterone (BSA-T), a BSA conjugated dihydrotestosteroneone (BSA-DHT) or both; and
      one or more optional pharmaceutically acceptable excipients, wherein the composition simultaneously kills one or more glioblastoma multiforme cells and protects one or more brain cells, neurons or both.
44. The method of embodiment 43, wherein the composition is administered orally, intravenously, intramuscularly, subcutaneously, intracranially or by another suitable parenteral route.
45. The method of embodiment 43, wherein the TMZ is administered in a dose ranging from 10 μM-10 mM and salinomycin is administered in a dose ranging from about 5 μM-10 mM.
46. The method of embodiment 43, wherein the BSA-T, BSA-DHT or both are administered in a dose ranging from 1 nM-10 μM.
47. The method of embodiment 43, wherein the BSA-T, BSA-DHT or both are administered prior to, concurrently or after the administration of TMZ.
48. A method of enhancing efficacy of a chemotherapy and in a subject suffering from glioblastoma multiforme comprising the step of: administering a bovine serum albumin (BSA) conjugated testotsterone (BSA-T), a BSA conjugated dihydrotestosteroneone (BSA-DHT) or both, wherein the BSA-T, BSA-DHT or both agents enhance a cytotoxic activity of temozolomide (TMZ), salinomycin or a combination of TMZ and salinomycin against the glioblastoma multiforme by simultaneously killing the one or more glioblastoma multiforme cells and protecting one or more brain cells, neurons or both.
49. The method of embodiment 48, wherein the BSA-T, BSA-DHT or both suppresses methylguanine methyltransferase (MGMT) activity, PI3K/Akt activity, extracellular signal regulated kinases (ERKs) or combinations thereof in the one or more glioblastoma multiforme cells.
50. The method of embodiment 48, wherein the composition is administered orally, intravenously, intramuscularly, subcutaneously, intracranially or by another suitable parenteral route.
51. The method of embodiment 48, wherein the TMZ is administered in a dose ranging from 10 μM-10 mM and salinomycin is administered in a dose ranging from about 5 μM-10 mM.
52. The method of embodiment 48, wherein the BSA-T, BSA-DHT or both are administered in a dose ranging from 1 nM-10 μM.
53. The method of embodiment 48, wherein the BSA-T, BSA-DHT or both are administered prior to, concurrently or after the administration of TMZ, salinomycin or a combination of TMZ, and salinomycin.
54. A method of enhancing efficacy of a radiation therapy in a subject suffering from glioblastoma multiforme comprising the step of: administering a composition comprising bovine serum albumin (BSA) conjugated testosterone (BSA-T), a BSA conjugated dihydrotestosteroneone (BSA-DHT) or both, wherein the BSA-T, BSA-DHT or both agents enhance a cytotoxic activity of ionizing radiation in the subject and, optionally, administering ionizing radiation to said subject.
55, The method of embodiment 54, wherein the composition is administered orally, intravenously, intramuscularly, subcutaneously, intracranially or by another suitable parenteral route.
56. The method of embodiment 54, further comprising the administration of a chemotherapeutic agent to said subject.
57. The method according to embodiment 56, the chemotherapeutic agent is administered prior to, concurrently or after the administration of said composition or is administered prior to, concurrently with or after treatment of the subject with ionizing radiation.
58. The method according to embodiment 57, wherein the chemotherapeutic agent is TMZ, salinomycin or a combination of TMZ and salinomycin.
59. The method of embodiment 58, wherein the TMZ is administered in a dose ranging from 10 μM-10 mM and salinomycin is administered in a dose ranging from about 5 μM-10 mM.
60. A method for enhancing simultaneously the efficacy of radiation therapy and for protecting one or more brain cells, neurons or both in a subject comprising the steps of:
   identifying the subject suspected of having a need for the treatment of a glioma; administering one or more membrane androgen receptor (mAR) activating agents, agonists or both, wherein the mAR agents enhance a cytotoxic activity of ionizing radiation on the glioma.
61. The method of embodiment 60, wherein the glioma is glioblastoma multiforme.
62. The method of embodiment 60, further comprising the administration of a chemotherapeutic agent.
63. The method of embodiment 62, wherein the chemotherapeutic agent is temozolomide or salinomycin.
64. The method of embodiment 60, wherein the one or more mAR activating agents or agonists selected from the group consisting of testosterone, dihydrotestosterone, methyltestosterone, active metabolites of testosterone. synthetic derivatives of testosterone, C-19 steroids with a side chain at C-17 and two angular methyl groups, and all androgenic derivatives of cyclopentanoperhydrophenanthrene.
65. The method of embodiment 60, wherein the mAR activating agent is selected from a testosterone or a dihydrotestosterone.
66. The method of embodiment 60, wherein the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle.
67. The composition of embodiment 66, wherein the conjugating agent is a protein selected from a bovine serum albumin or a human serum albumin.
68. The method of embodiment 60, wherein the mAR activating agent, agonists or both are administered orally, intravenously, intramuscularly, subcutaneously, intracranially or by another suitable parenteral route.
69. The method of embodiment 60, wherein the mAR activating agents, agonists or both are administered prior to, concurrently or after the radiation treatment.
70. The method according to embodiment 62 or 63, wherein the chemotherapeutic agent is administered prior to, concurrently or after the administration of said mAR containing composition or is administered prior to, concurrently with or after treatment of the subject with ionizing radiation.
71. The method of embodiment 63, wherein the TMZ is administered in a dose ranging from 10 μM-10 mM and salinomycin is administered in a dose ranging from about 5 μM-10 mM.
72. A method of treating, ameliorating symptoms, delaying progression or combinations thereof of one or more glial cancers in subject comprising the steps of:
   identifying the subject in need of the treatment, amelioration of the symptoms, delaying the progression or combinations thereof of the glial cancers; and
   administering a therapeutically effective amount of a pharmaceutical composition comprising one or more membrane androgen receptor (mAR) activating agents, agonists or both wherein the agents are selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene;
   and one or more optional pharmaceutically acceptable excipients, wherein the composition simultaneously kills one or more glial cancer cells and protects one or more brain cells, neurons or both; and administering radiation treatment to said subject.
73. The method of embodiment 72, wherein the glial cancers are selected from the group consisting of astrocytomas, ependymal tumors, glioblastoma multiforme, and primitive neuroectodermal tumors.
74. The method of embodiment 72, wherein the mAR activating agent, agonist or both are selected from a testosterone or a dihydrotestosterone.
75. The method of embodiment 72, wherein the synthetic derivatives of testosterone comprise testosterone propionate, testosterone cypionate, and fluoxymesterone.

76. The method of embodiment 72, wherein the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle.
77. The method of embodiment 76, wherein the conjugating agent is a protein selected from a bovine serum albumin or a human serum albumin.
78. The method of embodiment 72, wherein the composition is administered orally, intravenously, intramuscularly, subcutaneously, intracranially or another suitable parenteral route.
79. The method of embodiment 72, further comprising the administration of a chemotherapeutic agent.
80. The method according to embodiment 79, the chemotherapeutic agent is administered prior to, concurrently or after the administration of said composition or is administered prior to, concurrently with or after treatment of the subject with ionizing radiation.
81. The method according to embodiment 79 or 80, wherein one or more chemotherapeutic agent selected from the group consisting of dacarbazine alkylating agents, salinomycin, temozolomide, procarbazine, nitrosoureas, bis-chloronitrosourea, lomustine, and platinum based chemotherapeutic agents are administered to the subject.
82. The method according to embodiment 81, wherein the chemotherapeutic agent is TMZ, salinomycin or a combination of TMZ and salinomycin.
83. The method of embodiment 82, wherein the TMZ is administered in a dose ranging from 10 μM-10 mM and salinomycin is administered in a dose ranging from about 5 μM-10 mM.
84. The method of embodiment 72, wherein the one or more mAR activating agents or agonists are administered in a dose ranging from 1 nM-10 μM.
85. The method of embodiment 72, wherein said composition comprising one or more membrane androgen receptor (mAR) activating agents, agonists or both is administered prior to, concurrently or after the administration of said composition or is administered prior to, concurrently with or after treatment of the subject with ionizing radiation.
86. The method of embodiment 72, wherein said composition comprising one or more membrane androgen receptor (mAR) activating agents, agonists or both is administered prior to, concurrently or after the administration of said composition or is administered prior to, concurrently with or after treatment of the subject with ionizing radiation and/or one or more chemotherapeutic agent.
87, The method according to embodiment 86, wherein one or more chemotherapeutic agent selected from the group consisting of dacarbazine alkylating agents, salinomycin, temozolomide, procarbazine, nitrosoureas, bis-chloronitrosourea, lomustine, and platinum based chemotherapeutic agents are administered to the subject.
88. The method according to embodiment 87, wherein the chemotherapeutic agent is TMZ, salinomycin or a combination of TMZ and salinomycin.
89. The method of embodiment 88, wherein the TMZ is administered in a dose ranging from 10 μM-10 mM and salinomycin is administered in a dose ranging from about 5 μM-10 mM.
90. The method according to any one of embodiments 54-89, wherein said one or more membrane androgen activating agents is administered in a dose ranging from 1 nM-10 μM or a dose ranging from 1 μM-10 μM.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB, Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

United States Patent Application No. 20100048676 (Chang, 2010): Non-androgen dependent roles for androgen receptor in liver cancer.
United States Patent Application No. 20070141581 (Singh and Gatson, 2007): Membrane androgen receptor as a therapeutic target for the prevention/promotion of cell death.
United States Patent Application No. 20080267875 (Castanas, 2008): Steroid conjugates, preparation thereof and the use thereof.
Akhavan D, Cloughesy I F, Mischel P S (2010) mTOR signaling in glioblastoma: lessons learned from bench to bedside. Neuro Oncol. 12:882-9.
Augustine C K, Yoo J S, Potti A, Yoshimoto Y, Zipfel P A, Friedman H S, Nevins J R, Ali-Osman F, Tyler D S (2009) Genomic and molecular profiling predicts response to temozolomide in melanoma. Clin Cancer Res 15:502-510.
Braun A M, Thomas P (2004) Biochemical characterization of a membrane androgen receptor in the ovary of the atlantic croaker (Micropogonias undulatus). Biol Reprod 71:146-155.
Chakrabarti I, Cockburn M, Cozen W, Wang Y P, Preston-Martin S (2005) A population-based description of glioblastoma multiforme in Los Angeles County, 1974-1999. Cancer 104:2798-2806.
Chakravarti A, Zhai G, Suzuki Y, Sarkesh S, Black P M, Muzikansky A, Loeffler J S (2004) The prognostic significance of phosphatidylinositol 3-kinase pathway activation in human gliomas. J Clin Oncol 22:1926-1933.
Cheng C K, Fan Q W, Weiss W A (2009) P13K signaling in glioma--animal models and therapeutic challenges. Brain Pathol 19:112-120.
Clarke J, Butowski N, Chang S (2010) Recent advances in therapy for glioblastoma. Arch Neurol 67:279-283.
Curran W J, Jr., Scott C B, Horton J, Nelson J S, Weinstein A S, Fischbach A J, Chang C H, Rotman M, Asbell S O, Krisch R E, et al. (1993) Recursive partitioning analysis of prognostic factors in three Radiation Therapy Oncology Group malignant glioma trials. J Natl Cancer Inst 85:704-710.
Dudek H, Datta S R, Franke T F, Birnbaum M J, Yao R, Cooper G M, Segal R A, Kaplan D R, Greenberg M E (1997) Regulation of neuronal survival by the serine-threonine protein kinase Akt. Science 275:661-665.
Erlanger B F, Borek F, Beiser S M, Lieberman S (1957) Steroid-protein conjugates. I. Preparation and characterization of conjugates of bovine serum albumin with testosterone and with cortisone. J Biol Chem 228:713-727.
Franke T F, Kaplan D R, Cantley L C, Toker A (1997) Direct regulation of the Akt proto-oncogene product by phosphatidylinositol-3,4-bisphosphate. Science 275:665-668.
Friedman H S, Kerby T, Calvert H (2000) Temozolomide and treatment of malignant glioma. Clin Cancer Res 6:2585-2597.
Fujiwara K, Iwado E, Mills G B, Sawaya R, Kondo S, Kondo Y (2007) Akt inhibitor shows anticancer and radiosensitizing effects in malignant glioma cells by inducing autophagy. Int J Oncol 31:753-760.
Grossman S A, Batara J F (2004) Current management of glioblastoma multiforme. Semin Oncol 31:635-644.
Hatzoglou A, Kampa M, Kogia C, Charalampopoulos I, Theodoropoulos P A, Anezinis P, Dambaki C, Papakonstanti E A, Stathopoulos E N, Stournaras C, Gravanis A, Castanas E. (2005) Membrane androgen receptor activation induces apoptotic regression of human prostate cancer cells in vitro and in vivo. J. Clin. Endocrinol. Metab. 90:893-903.
Holland E C, Celestino J, Dai C, Schaefer L, Sawaya R E, Fuller G N (2000) Combined activation of Ras and Akt in neural progenitors induces glioblastoma formation in mice. Nat Genet 25:55-57.
Jacques-Silva M C, Bernardi A, Rodnight R, Lenz G (2004) ERK, PKC and PI3K/Akt pathways mediate extracellular ATP and adenosine-induced proliferation of U138-MG human glioma cell line. Oncology 67:450-459.
Kaina B, Christmann M, Naumann S, Roos WP (2007) MGMT: key node in the battle against genotoxicity, carcinogenicity and apoptosis induced by alkylating agents. DNA Repair (Amst) 6:1079-1099.
Kampa M, Kogia C, Theodoropoulos P A, Anezinis P, Charalampopoulos I, Papakonstanti E A, Stathopoulos E N, Hatzoglou A, Stournaras C, Gravanis A, Castanas E. (2006) Activation of membrane androgen receptors potentiates the antiproliferative effects of paclitasel on human prostate cancer cells. Mol Cancer Ther 5:1342-51.
Mangelsdorf D J, Thummel C, Beato M, Herrlich P, Schutz G, Umesono K, Blumberg B, Kastner P, Mark M, Chambon P, et al. (1995) The nuclear receptor superfamily: the second decade. Cell 83:835-839.
Mason W P, Cairncross J G (2005) Drug Insight: temozolomide as a treatment for malignant glioma—impact of a recent trial. Nat Clin Pract Neurol 1:88-95.
Maxwell J A, Johnson S P, McLendon R E, Lister D W, Home K S, Rasheed A, Quinn J A, Ali-Osman F, Friedman A H, Modrich P L, Bigner D D, Friedman H S (2008) Mismatch repair deficiency does not mediate clinical resistance to temozolomide in malignant glioma. Clin Cancer Res 14:4859-4868.
Minniti G, Muni R, Lanzetta G, Marchetti P, Enrici R M (2009) Chemotherapy for glioblastoma: current treatment and future perspectives for cytotoxic and targeted agents. Anticancer Res 29:5171-5184.
Ohgaki H, Dessen P, Jourde B, Horstmann S, Nishikawa T, Di Patre P L, Burkhard C, Schuler D, Probst-Hensch N M, Maiorka P C, Baeza N, Pisani P, Yonekawa Y, Yasargil M G, Lutolf U M, Kleihues P (2004) Genetic pathways to glioblastoma: a population-based study. Cancer Res 64:6892-6899.
Papadopoulou N, Charalampopoulos I, Anagnostopoulou V, Konstantinidis G, Foller M, Gravanis A, Alevizopoulos K, Lang F, Stournaras C (2008) Membrane androgen receptor activation triggers down-regulation of PI-3K/Akt/NF-kappaB activity and induces apoptotic responses via Bad, FasL and caspase-3 in DU145 prostate cancer cells. Mol Cancer 7:88.
Rajasekhar V K, Viale A, Socci N D, Wiedmann M, Hu X, Holland E C (2003) Oncogenic Ras and Akt signaling contribute to glioblastoma formation by differential recruitment of existing mRNAs to polysomes. Mol Cell 12:889-901.
Sonoda Y, Ozawa T, Aldape K D, Deen D F, Berger M S, Pieper R O (2001) Akt pathway activation converts anaplastic astrocytoma to glioblastoma multiforme in a human astrocyte model of glioma. Cancer Res 61:6674-6678.
Suzuki Y, Shirai K, Oka K, Mobaraki A, Yoshida Y, Noda SE, Okamoto M, Itoh J, Itoh H, Ishiuchi S, Nakano T (2010) Higher pAkt expression predicts a significant worse prognosis in glioblastomas. J Radiat Res (Tokyo) 51:343-348.
Wrensch M, Minn Y, Chew T, Bondy M, Berger M S (2002) Epidemiology of primary brain tumors: current concepts and review of the literature. Neuro Oncol 4:278-299.
Yin L T, Fu Y J, Xu Q L, Yang J, Liu Z L, Liang A I I, Fan X J, Xu C G (2007) Potential biochemical therapy of glioma cancer. Biochem Biophys Res Commun 362:225-229.
Yoshino A, Ogino A, Yachi K, Ohta T, Fukushima T, Watanabe T, Katayama Y, Okamoto Y, Naruse N, Sano E, Tsumoto K (2010) Gene expression profiling predicts response to temozolomide in malignant gliomas. Int J Oncol 36:1367-1377.
Zhang J, Stevens M F, Laughton C A, Madhusudan S, Bradshaw T D (2010) Acquired resistance to temozolomide in glioma cell lines: molecular mechanisms and potential translational applications. Oncology 78:103-114.
Zheng J, Ali A, Ramirez V D (1996) Steroids conjugated to bovine serum albumin as tools to demonstrate specific steroid neuronal membrane binding sites. J Psychiatry Neurosci 21:187-197.

We claim:

1. A composition for enhancing simultaneously the effectiveness of one or more chemotherapeutic agents and for protecting one or more brain cells, neurons or both, wherein the chemotherapeutic agents treat, ameliorate symptoms, or delay a progression of one or more gliomas comprising:
one or more chemotherapeutic agents selected from the group consisting of dacarbazine alkylating agents, salinomycin, temozolomide, procarbazine, nitrosoureas, bis-chloronitrosourea, lomustine, and platinum based chemotherapeutic agents;
one or more membrane androgen receptor (mAR) activating agents, agonists or both, wherein the agents are selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene; and
one or more optional pharmaceutically acceptable excipients.

2. The composition of claim 1, wherein the mAR activating agent is selected from a testosterone or a dihydrotestosterone.

3. The composition of claim 1, wherein the synthetic derivatives of testosterone comprise testosterone propionate, testosterone cypionate, and fluoxymesterone.

4. The composition of claim 1, wherein the mAR activating agent, agonist or both further comprise a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle.

5. The composition of claim 4, wherein the conjugating agent is a protein selected from a bovine serum albumin or a human serum albumin.

6. The composition of claim 1, wherein the composition is administered orally, intravenously, intramuscularly, subcutaneously, intracranially or by another suitable parenteral route.

7. The composition of claim 1, wherein said composition comprises one or more chemotherapeutic agents in a dose ranging from 10 μM-10 mM.

8. The composition of claim 1, wherein said composition comprises one or more mAR activating agents or agonists in a dose ranging from 1 nM-10 μM.

9. The composition of claim 1, wherein the chemotherapeutic agent is TMZ, salinomycin or a combination of TMZ and salinomycin.

10. A method of treating, ameliorating symptoms, delaying progression or combinations thereof of one or more glial cancers in subject comprising the steps of:
identifying the subject in need of the treatment, amelioration of the symptoms, delaying the progression or combinations thereof of the glial cancers; and
administering a therapeutically effective amount of a pharmaceutical composition sufficient to treat, ameliorate symptoms, delay progression or combinations thereof of the one or more cancers in the subject comprising:
one or more chemotherapeutic agents, wherein the one or more chemotherapeutic agents are selected from the group consisting of dacarbazine alkylating agents, salinomycin, temozolomide, procarbazine, nitrosoureas, bis-chloronitrosourea, lomustine, and platinum based chemotherapeutic agents;
one or more membrane androgen receptor (mAR) activating agents, agonists or both wherein the agents are selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene; and
one or more optional pharmaceutically acceptable excipients, wherein the composition simultaneously kills one or more glial cancer cells and protects one or more brain cells, neurons or both.

11. The method of claim 10, wherein the glial cancers are selected from the group consisting of astrocytomas, ependymal tumors, glioblastoma multiforme, and primitive neuroectodermal tumors.

12. A method for enhancing simultaneously the efficacy of a chemotherapy and for protecting one or more brain cells, neurons or both in a subject comprising the steps of:
identifying the subject suspected of having a need for the treatment of a glioma; and
administering one or more membrane androgen receptor (mAR) activating agents, agonists or both, wherein the mAR agents enhance a cytotoxic activity of the one or more chemotherapeutic agents.

13. The method of claim 12, wherein the glioma is glioblastoma multiforme.

14. The method of claim 12, wherein the chemotherapeutic agent is temozolomide (TMZ), salinomycin or a combination of TMZ and salinomycin.

15. A method of treating, ameliorating symptoms, delaying progression or combinations thereof of glioblastoma multiforme in a subject comprising the steps of:
identifying the subject in need of the treatment, amelioration of the symptoms, delaying progression or combinations thereof of the glioblastoma multiforme; and
administering a therapeutically effective amount of a pharmaceutical composition sufficient to treat or ameliorate the symptoms of the one or more cancers in the subject comprising:
temozolomide (TMZ), salinomcyin or a combination of TMZ and salinomycin;
a bovine serum albumin (BSA) conjugated testosterone (BSA-T), a BSA conjugated dihydrotestosteroneone (BSA-DHT) or both; and
one or more optional pharmaceutically acceptable excipients, wherein the composition simultaneously kills one or more glioblastoma multiforme cells and protects one or more brain cells, neurons or both.

16. The method of claim 15, wherein the composition is administered orally, intravenously, intramuscularly, subcutaneously, intracranially or by another suitable parenteral route.

17. A method of treating, ameliorating symptoms, delaying progression or combinations thereof of one or more glial cancers in subject comprising the steps of:
identifying the subject in need of the treatment, amelioration of the symptoms, delaying the progression or combinations thereof of the glial cancers; and
administering a therapeutically effective amount of a pharmaceutical composition comprising one or more membrane androgen receptor (mAR) activating agents, agonists or both wherein the agents are selected from the group consisting of testosterone, dihydrotestosterone, methyltestosterone, active metabolites of testosterone, synthetic derivatives of testosterone, C-19 steroids with a side chain at C-17 and two angular methyl groups, and all androgenic derivatives of cyclopentanoperhydrophenanthrene;
and one or more optional pharmaceutically acceptable excipients, wherein the composition simultaneously kills one or more glial cancer cells and protects one or more brain cells, neurons or both; and
administering radiation treatment to said subject.

18. The method of claim 17, wherein the glial cancers are selected from the group consisting of astrocytomas, ependymal tumors, glioblastoma multiforme, and primitive neuroectodermal tumors.

19. The method of claim 17, wherein the mAR activating agent, agonist or both are selected from a testosterone or a dihydrotestosterone.

20. The method of claim 17, wherein the synthetic derivatives of testosterone comprise testosterone propionate, testosterone cypionate, and fluoxymesterone.

21. The method of claim 17, wherein the mAR activating agent, agonist or both is defined further as comprising a conjugating agent, wherein the conjugating agent is selected from the group consisting of a bead, a large protein, a nucleic acid, a lipid, a fatty acid, a carbohydrate, a charged molecule, a glass, a quartz, a silicon, a polymer, a multimer, an oligomer, a metal, a nanoparticle, and a microparticle.

22. The method of claim 21, wherein the conjugating agent is a protein selected from a bovine serum albumin or a human serum albumin.

23. The method of claim 17, wherein the composition is administered orally, intravenously, intramuscularly, subcutaneously, intracranially or another suitable parenteral route.

24. The method of claim 17, further comprising the administration of a chemotherapeutic agent.

25. The method of claim 24, the chemotherapeutic agent is administered prior to, concurrently or after the administration of said composition or is administered prior to, concurrently with or after treatment of the subject with ionizing radiation.

26. The method according to claim 24, wherein one or more chemotherapeutic agent selected from the group consisting of dacarbazine alkylating agents, salinomycin, temozolomide, procarbazine, nitrosoureas, bis-chloronitrosourea, lomustine, and platinum based chemotherapeutic agents are administered to the subject.

27. The method of claim 26, wherein the chemotherapeutic agent is TMZ, salinomycin or a combination of TMZ and salinomycin.

28. The method of claim 17, wherein said composition comprising one or more membrane androgen receptor (mAR) activating agents, agonists or both is administered prior to, concurrently or after the administration of said composition or is administered prior to, concurrently with or after treatment of the subject with ionizing radiation.

29. The method of claim 17, wherein said composition comprising one or more membrane androgen receptor (mAR) activating agents, agonists or both is administered prior to, concurrently or after the administration of said composition or is administered prior to, concurrently with or after treatment of the subject with ionizing radiation and/or one or more chemotherapeutic agent.
